(19) United States
(12) Patent Application Publication
(10) Pub. No.: US 2006/0051224 A1
(43) Pub. Date: Mar. 9, 2006

(54) Pusher fire pumper tanker (The cannon pump)

(76) Inventor: Majed Mohammed H. Alsubiei, (US)

Correspondence Address: Majed Mohammed H. Alsubiei, 3230 San Francisco Ave., Long Beach, CA, 90806, US

(21) Appl. No.: 11/214,094

(22) Filed: Aug. 30, 2005

(30) Foreign Application Priority Data
Sep. 6, 2004 (SA) 04250278

(51) Int. Cl. A61M 1/00

(52) U.S. Cl. 417/437

(57) ABSTRACT

A fire extinguisher pumper tanker-a cylinder tank 1, includes extinguishing liquids filling in pipes 21, & combustion chamber 2 pushed piston 50 by burning force until the piston hits & stopped by the surround front cover 4, the piston push the extinguishing liquids, throw the buffer object 36, throw the front cover 3 & throw the barrel-nozzle 5, the tanker pump guided by six (or more) hydraulic cylinders 7-8-9 by lifting the pump from front or rear or both sides, the pusher piston will return back by multi layers gas cylinders 11, & the pump will suck-filling up liquids again.

This invention relates to fire engines-fire pampers & tankers.

Now days fire pumps had a lot of disadvantages, those now days pumps-tankers working by propellers moved by engines, those pumps suck water from side then push it from the other side, or tankers pumps by compressed air-gas pump, these pumps had a lot of small parts, its pumps continues water in low quantities, the water-extinguishing liquids will evaporate quick if the fire is very hot-very big, as like gas-oil wells fires, gas-oil fields fires, oil-gas pips fires chemical-petrochemical factories fires & forests-jungles fires, they must changed parts to pumps deferent kinds of liquids, they cant pumps from-to far distances, as like sky scrapers fires & military warehouses its very dangerous to fire men to came near them, because the skyscraper may collapse or the warehouse explode, The fire men needs to come near by (close as possible) to the fire source because there anti-fire pumps can not pumping the water (liquids) to the fire source from far distance. Every time firemen come nearer to the fire the dangerous increases on them,

But the cannon pump is a very powerful anti fire pump, it can pump from-to far distances, & it can pumps huge amounts of liquids-fluids in tiny time. The cannon pump used to extinguish all kinds of tall buildings (skyscrapers) fires, oil-gas fields, wells & pipes fires, chemicals-petrochemicals factories fires, arms-munitions-bombs-biochemical's warehouses fires & forests-jungles fires, & its provides safety to the fire men, because its can pumps from-to far distances.

The invention will now be described solely by way of example and with reference to the accompanying drawings in which:

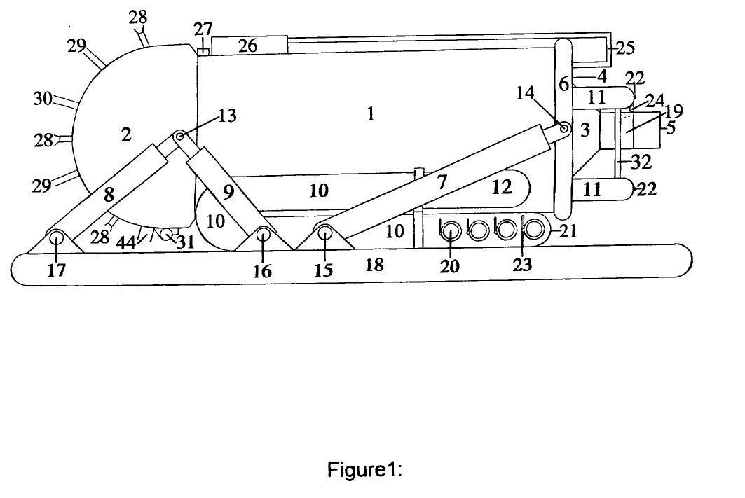

FIG. 1 shows cannon pump outer side,

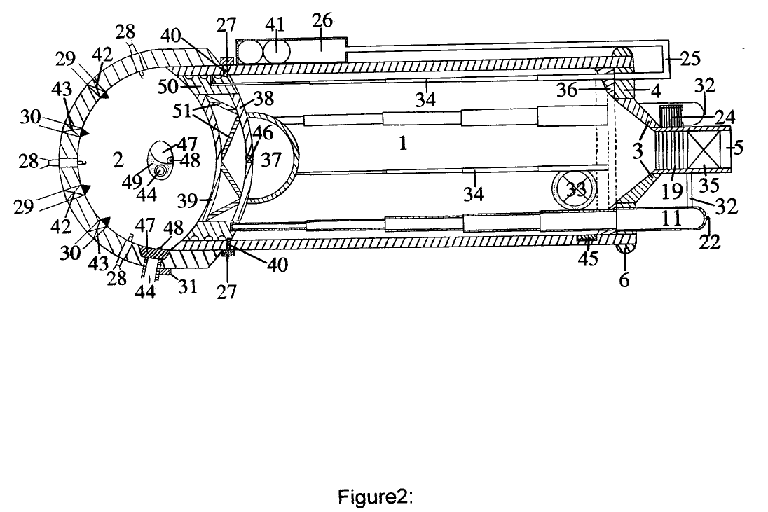

FIG. 2 shows cannon pump main body-the pump internal parts,

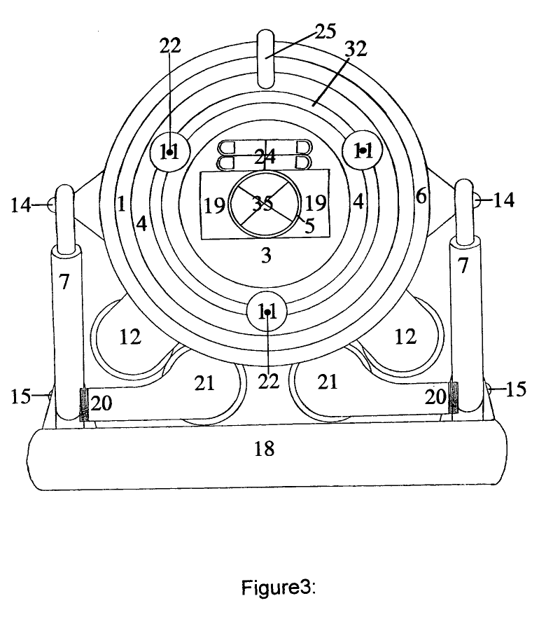

FIG. 3 shows cannon pump front side,

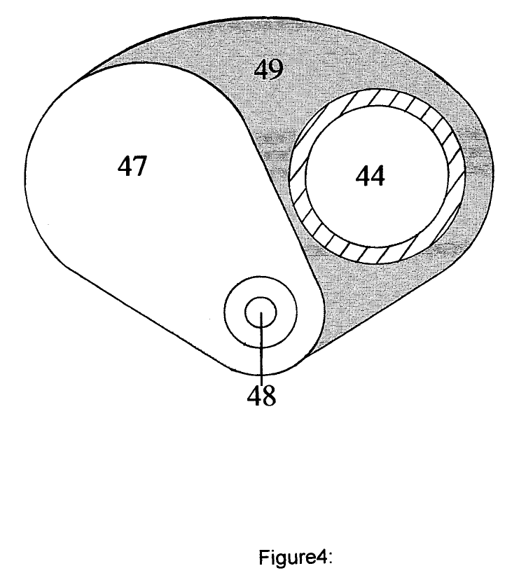

FIG. 4 shows the rotate able valve front side,

FIGS. 1,2 & 3, shows the cannon pump all parts, the cannon pump includes a cylinder tank 1 (or semi cylinder tank), & there are two (or more) holes in each side of the cylinder tank, in position before the buffer-absorber object 36, those two holes 33 includes one way valves 33, & there are two hard pipes 12 steadied to those two holes 33, & there are two flexible pipes 10 moving with the pump movement steadied to those two hard pipes 12 from other end in both side, & those two flexible pipes 10 other ends steadied to two liquid fill in hard pipes 21 in both sides, in each liquid fill in hard pipe 21 there are a lot of water fill-in connect holes 20, each fluids fill in connect hole 20 includes a manual two way valve 23, the cannon pump includes ring shape frame 6 steadied around the cylinder tank 1 out front surface, the ring shape frame 6 inner surface conforms with the cylinder tank 1 out front surface, there are two holders arms 14 steadied in the ring shape frame 6 in both sides, there are two hydraulic cylinders 7 steadied to them in both sides, the cannon pump includes cylinder-half ball shape (or semi cylinder-semi half ball shape) combustion chamber 2 steadied to the cylinder tank rear, the combustion chamber 2 contained fuel 30 air 29 supplying pipes coming from air-fuel pumps, the air 29 fuel 30 pipes includes one way valve for each, those valves allowed air-fuel to come into the combustion chamber 2 from air-fuel pumps, the combustion chamber also includes quarter circle shape (or semi quarter circle shape) rotate able valves 47, the rotate able valves rotate in half a circle shape wide cannel 49 in combustion chamber 2 internal surface, because this cannel will not allowed rotate able valves 47 to rotate in fully circle & to allowed them rotate into two directions, direction for closing the exhaust tube 44 in charging in combustion chamber air & fuel, & another fill out the exhaust gas, the rotate able valves moves by to ways moving electric motors 31 steadied on combustion chamber 2 outer surface, these electric motors 31 rotate pillars 48 went cross the combustion chamber 2 body & steadied to the rotate able valves 47 to moves them (see FIG. 4), & there is spark plugs steadied to combustion chamber for starting burning the combustion, the cannon pump includes also ring shape pusher piston 50, moving inside the cylinder tank 1, the piston 50 out surface conform with the cylinder tank 1 inside surface, there is a front cover 38 steadied to the piston front, & there is a rear cover 39 steadied to the piston rear, & there are support props 51 between the two piston covers, there is a gas balloon 37 steadied to piston front cover 38 & it had a valve 46 for fill gas in, this gas balloon 37 absorb the combustion burning starting force, the pusher piston 50 moves from combustion chamber to rear until its hits the buffer-absorber object 36, the pusher piston come back again to rear in filling liquids operation, because it will be pushed back by the gas multi layers cylinders 11, & because the gravity force caused by the two front hydraulic cylinders 7 lifting the pump from up, the cannon pump includes pusher piston buffer-absorber object 36, steadied to rear the front surround cover 4 for prevent the pusher piston damages the front surround cover 4 when the piston 50 hits it, the buffer object 36 semi funnel-conical ring shape, coming more tight to front, it steadied to the surround front cover 4, its allowing pushed water to go throw it & throw the cylinder tank front cover 3, the cannon pump includes front cover 3 steadied to the surround front cover 4, the front cover 3 funnel-conical shape, the hole in it becoming more tight to front, & steadied to this hole barrel-nozzle 5, the cannon pump includes barrel-nozzle 5 contained air turbine 19 charger 24, & one way valve 35, this valve 35 allowing air & liquids go out throw the barrel-nozzle 5, but not allowing air come inside the tanker cylinder when tanker cylinder filling in liquids, the cannon pump includes six (or more) lifters-guiders hydraulic cylinders 7-8-9 in both sides, guiding pump to target, the front two hydraulic cylinders 7 steadied into two arms 14 steadied to the front ring frame 6, the four rear hydraulic cylinders 8-9 steadied into two arms 13 steadied to combustion chamber 2 outer surface, all the hydraulic cylinders 7-8-9 other ends steadied into six arms 15-16-17 steadied to the base 18, the hydraulic cylinders works by oil-gas pressures for lifting up-down the cannon from front-rear or both sides, to guide the pump & for absorb the first momentum caused by the fuel burn in combustion chamber 2, & also to absorb the second momentum caused by the pusher piston 50 hits the buffer object 36, the cannon pump includes three (or more) multi layers gas cylinders 11 contains pressed gas, steadied to the pusher piston 50, & steadied to surround front cover 4 from the other end, it will push back the piston 50 after the combustion chamber 2 exhaust tubes opens 44, the cannon pump includes piston oiling multi layers cylinders 34, steadied to the surround front cover from end & to the piston 50 in the other end, the oil passing throw it from the oil tank 26 that contains gas balloons 41 to push the oil to the pusher piston 50 to oil it, the cannon pump includes base 18 steadied on it the cannon pump, the base 18 contained fuel-air tanks pumped by fuel-air pumps, & contained hydraulic oil used in hydraulic cylinders 7-8-9, & hydraulic pumps, the cannon pump includes also the automatic controller switch 45, when this switch pressed by the piston it will give an order to the electric motors 31 to open the exhaust tubes 44, by rotate the rotate able valves 47, the cannon pump includes also piston grabbers-holders 40 moved by electric motors 27, to grabs-holds the piston 50 in it position while the combustion chamber 2 filling in air & fuel, & to let the pusher piston 50 lose free before-when the spark plugs 28 works.

1. The pusher fire pumper tanker (the cannon pump) included from:
a cylinder tank (or semi cylinder tank), & there are two (or more) holes in each side of the cylinder, in position before the buffer-absorber object, those two holes includes one way valves, & there are two hard pipes steadied to those two holes, & there are two flexible pipes moving with the pump movement steadied to those two hard pipes in both side, & those two flexible pipes other ends steadied to two liquid fill in hard pipes in both sides, in each liquid fill in hard pipe includes a lot of water fill connect holes, each water fill in hard pipe includes a manual two way valve.

2. The pusher fire pumper tanker (the cannon pump) according to claim 1, the cannon pump includes ring shape frame steadied around the cylinder tank out front surface, the ring shape frame inner surface conforms with the cylinder tank out front surface, there are two holders arms steadied in the ring shape frame, in these two holders arms there are two hydraulic cylinders steadied to them.

3. The pusher fire pumper tanker (the cannon pump) according to claim 2, the cannon pump includes cylinder-half ball shape (or semi cylinder-semi half ball) combustion chamber steadied to the cylinder tank rear, the combustion chamber contained fuel-air supplying pipes coming from air-fuel pumps, the air-fuel pipes includes one way valve for each, those valves allowed air-fuel to come into the combustion chamber from air-fuel pumps, the combustion chamber also includes quarter circle shape (or semi quarter circle shape) rotate able valves, the rotate able valves rotate in half a circle shape wide cannel in combustion chamber internal surface, because this cannel will not allowed rotate able valves to rotate in fully circle & to rotate into two directions, direction for closing the exhaust tube in charging in combustion chamber air & fuel, direction for opening the exhaust tube for fill out the exhaust gas, the rotate able valves moves by electric motors steadied on combustion chamber outer surface, these electric motors rotate pillars went cross the combustion chamber body & steadied to the rotate able valves to moves them, & there is spark plugs steadied to the combustion chamber for starting burning the combustion.

4. The pusher fire pumper tanker (the cannon pump) according to claim 3, the cannon pump includes ring shape pusher piston, moving inside the cylinder tank, the piston out surface conform with the cylinder tank inside surface, there is a front cover steadied to the piston front, & there is a rear cover steadied to the piston rear, & there are support props between the two piston covers, also there is a gas balloon steadied to piston front cover & it had a valve for filling gas in, this gas balloon absorb the combustion burning starting force, the pusher piston move from combustion chamber to rear until its hits the buffer-absorber object, the pusher piston come back again to rear in filling in time, because it will be pushed back by the gas multi layers cylinders, & because the gravity force because the two front hydraulic cylinders will lift the pump from up.

5. The pusher fire pumper tanker (the cannon pump) according to claim 4, the cannon pump includes pusher piston buffer-absorber object, steadied to inside the front surround cover for prevent the pusher piston damages the front surround cover when piston hits it, the buffer object semi funnel-conical ring shape, coming more tight to front, it steadied to the surround front cover, allowing pushed water to go throw it & throw the cylinder tank front cover.

6. The pusher fire pumper tanker (the cannon pump) according to claim 5, the cannon pump includes front cover steadied to the surround front cover, the front cover funnel-conical shape, the hole in it becoming more tight to front, steadied to this hole barrel-nozzle.

7. The pusher fire pumper tanker (the cannon pump) according to claim 6, the cannon pump includes barrel-nozzle contained air turbine charger, & one way valve, this valve allowing air & liquids go out the barrel, but not allowing air coming inside the tanker cylinder when tanker cylinder filling liquids in.

8. The pusher fire pumper tanker (the cannon pump) according to claim 7, the cannon pump includes six (or more) lifters-guiders
hydraulic cylinders in both sides, guiding pump to target, the front two hydraulic cylinders steadied into two arms steadied to the front ring frame,
the four rear hydraulic cylinders steadied into two arms steadied to combustion chamber outer surface, all the hydraulic cylinders another ends steadied into six arms (or more) steadied to the base, the hydraulic cylinders works by oil-gas pressures for lifting up-down the cannon pump from front-rear or both sides, to guide the pump & for absorb the first momentum caused by the fuel burn in combustion chamber, & also to absorb the second momentum caused by the pusher piston hits the buffer object.

9. The pusher fire pumper tanker (the cannon pump) according to claim 8, the cannon pump includes three (or more) multi layers gas cylinders contains pressed gas, steadied to the pusher piston, & steadied to surround front cover from the another end, it will push back the piston after the combustion chamber exhaust tubes opens.

10. The pusher fire pumper tanker (the cannon pump) according to claim 9, the cannon pump includes piston oiling multi layers
cylinders, steadied to the surround front cover from end & to the piston in the other end, the oil passing throw it from the oil tank that contains gas balloons to push the oil to the pusher piston to oil it.

11. The pusher fire pumper tanker (the cannon pump) according to claim 10, the cannon pump includes base steadied on top of it the cannon pump, the base contained fuel-air tanks pumped by fuel-air pumps, & contained hydraulic oil used in hydraulic cylinders, & hydraulic pumps.

12. The pusher fire pumper tanker (the cannon pump) according to claim 11, the cannon pump includes the automatic controller switch, when this electric switch pressed by the piston it will give an order to the electric motors to open the exhaust tubes, by rotate the rotate able valves.

13. The pusher fire pumper tanker (the cannon pump) according to claim 12, the cannon pump includes piston grabbers-holders moved by electric motors, to grabs-holds the piston in steady position while the combustion chamber charging in air & fuel, & they will lose it free when-before the spark plugs works.
